(19) United States
(12) Patent Application Publication
(10) Pub. No.: US 2011/0218416 A1
(43) Pub. Date: Sep. 8, 2011

(54) Needle Electrode Module

(76) Inventors: Ethan S. Lauer, Austin, TX (US); Patrick Hemstreet, Houston, TX (US)

(21) Appl. No.: 13/039,996

(22) Filed: Mar. 3, 2011

Related U.S. Application Data
(60) Provisional application No. 61/309,964, filed on Mar. 3, 2010.

(51) Int. Cl. A61B 5/04

(52) U.S. Cl. 600/377

(57) ABSTRACT

The invention is a needle electrode module, such as for use in neurodiagnostic monitoring, that helps minimize the risk of needle-stick injuries. As described, the needle electrode module includes a means for preventing the exposure of the distal end of a needle element until the module is placed against a surface. In an embodiment, the distal end of a needle element may be retracted into the needle electrode module when the needle electrode module is removed from the punctured surface and, once retracted, the distal end of the needle may be prevented from being accidentally re-exposed. Several embodiments use a mechanical mechanism to hold the distal end of the needle element in the retracted or exposed positions and a spring force to retract the distal end of the needle. Another embodiment uses suction to hold the distal end of the needle element in the exposed position and the force necessary to deform the body of the needle electrode module to retract the distal end of the needle element in the retracted position and maintain it in the retracted position.

### CROSS-REVERENCE TO RELATED APPLICATIONS

This patent claims the benefit of U.S. Provisional Application No. 61/309,964 under 35 U.S.C. §119 (e).

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

This invention relates generally to medical needles and the means for handling the same. In particular, the invention relates to a needle electrode module that prevents accidental exposure of the needle before and after use, but that, when placed against a surface, allows insertion of a needle electrode to a point below the surface. As such, the invention will minimize the risk of needle-stick injuries to persons operating the module.

The monitoring of nervous system electrical signals has been found to be beneficial during medical procedures that involve access to, manipulation of, or removal of areas of the body that include or are located near important nerves or nerve bundles. Damage to important nerves or nerve bundles may cause lasting or irreparable harm to a patient. Monitoring the electrical signals of the nervous system for changes may indicate to personnel involved in, directing or performing a medical procedure when there is an increased risk of imminent danger to an important nerve or nerve bundle so that the medical procedure may be stopped or altered to minimize such risk.

A method of monitoring nervous system electrical signals involves the use of at least one needle electrode that penetrates the surface of the skin such that the distal end of the needle electrode is located in proximity to a nerve or nerve bundle. Thus far, needle electrodes have generally comprised needles inserted through sheaths into the skin by hand. For example, such mechanisms are disclosed in U.S. Pat. Nos. 5,279,578 (Cooke), 4,928,824 (Barasch) and 7,308,318 B2 (Miazga, et al.). Barasch further teaches the use of a cap placed over the sheath after use to protect against post-use needle-stick accidents. Cooke and Miazga teach the manual withdrawal of the needle into the sheath to protect against post-use needle-stick accidents. None of the prior art prevent exposure of the needle prior to the location of the needle on the surface intended to be punctured. Nor does the prior art teach automatic retraction of a needle into a protective sheath or housing upon removal of the protective sheath or housing from a surface after use.

While the prior art teaches various methods for protecting medical personnel during the transport and insertion of needles, what is needed in the art is an apparatus that prevents exposure of a needle until the needle module is adjacent to a surface for use and that automatically retracts the needle when use is accidentally or intentionally terminated.

### BRIEF SUMMARY OF THE INVENTION

An object of the present invention is the provision of a needle electrode module that helps minimize the risk of needle-stick injuries by preventing exposure of the needle until the needle electrode module is placed against a surface. A further object is the provision of a needle electrode module that automatically retracts the needle when the module is removed from a surface.

### BRIEF DESCRIPTION OF THE SEVERAL VIEWS OF THE DRAWING

FIG. 1 is a sectional view of a first embodiment of the invention with the needle electrode in a retracted configuration.

FIG. 2 shows an exploded elevational view of the first embodiment of the invention.

FIG. 3 shows a sectional view of a second embodiment of the invention with the needle electrode in a retracted configuration.

FIG. 4 is an exploded elevational view of the second embodiment of the invention.

FIG. 5 is a sectional view of a third embodiment of the invention with the needle electrode in a retracted configuration.

FIG. 6 is an exploded elevational view of the third embodiment of the invention.

### DETAILED DESCRIPTION OF THE INVENTION

One way to assess the activity of a patient's nervous system is to monitor the electrical signals generated by the patient's nervous system. The electrical signals generated by a patient's nervous system may be detected by electrodes that extend through the patient's skin and other tissue to be in proximity of nerves or nerve bundles. Such needle electrodes have sharp distal ends to facilitate insertion through the skin and other tissue. As a result, electrodes may accidentally puncture the skin of caregivers prior to insertion in a patient or after removal from a patient. This invention provides a needle electrode module that prevents exposure of the needle until the needle electrode module is proximity of the surface to be punctured. Further, this invention provides for the automatic retraction of needle electrode after use.

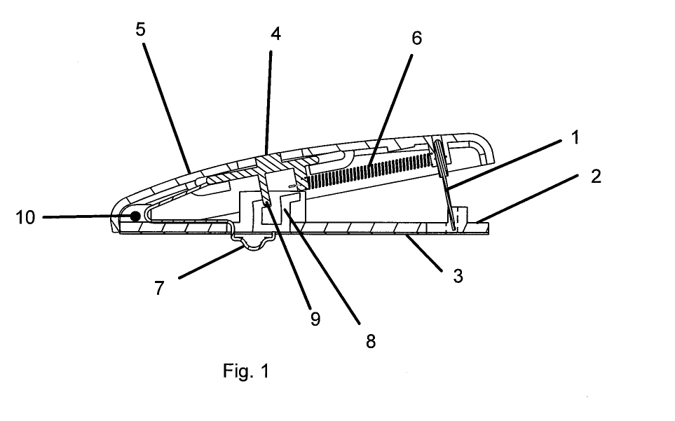

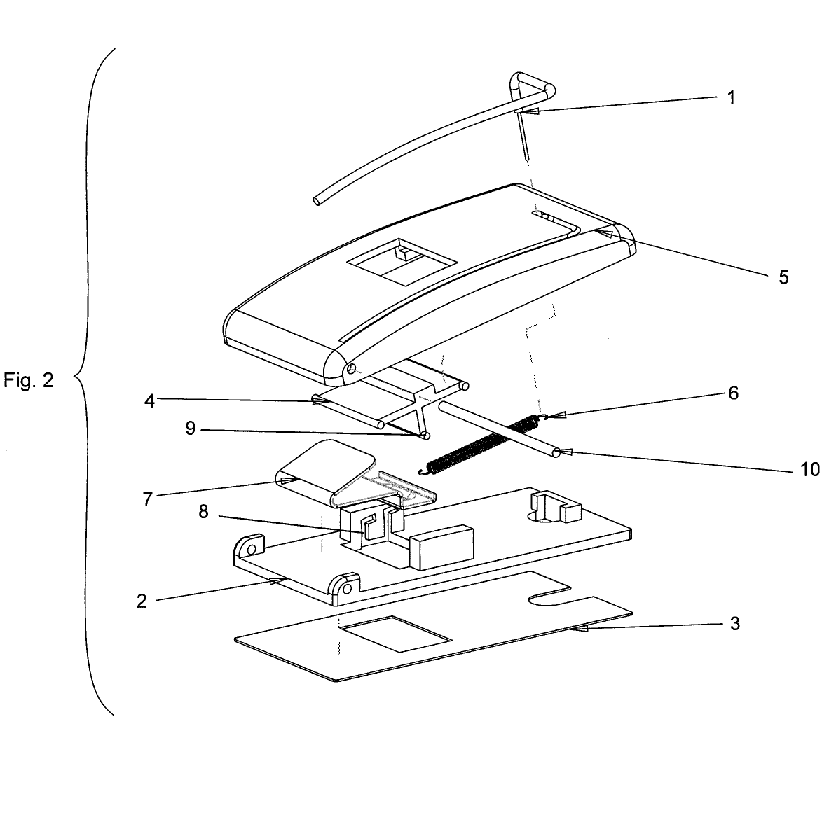

FIG. 1 shows a sectional view of a preferred embodiment of this invention with a needle member 1 in a first position in which the distal end of the needle member 1 does not extend below the plane of the base member 2. In a preferred embodiment of the invention shown in FIGS. 1-2 an adhesive layer 3, for example tape, is affixed to the portion of the base member 2 that is to be located in proximity to the surface to secure the base member 2 to the surface. The invention further comprises a means for securing the needle member 1 in its first position when the base member 2 is not in the proximity of the surface and allowing the needle member 1 to move into and stay in its second position when the base member 2 is in the proximity of the surface.

As shown in FIGS. 1-2, in a preferred embodiment, the means for securing the needle member 1 as described above comprises a first member 4 and second member 5 wherein the first member 4 and second member 5 each have at least a first and second position. As shown in FIG. 1, in a preferred embodiment, when the first member 4 is in its first position the second member 5 cannot move from its first position. When the first member 4 is in its second position, the second member 5 may move to its second position and cause the needle member 1 to move to a second position in which the distal end of the needle member 1 extends below the plane of the base member 2. Also, a preferred embodiment comprises a means for exerting a force on the first member 4 that tends to move the first member 4 from its second position to its first position. As shown in FIGS. 1-2, the means for exerting the force on the first member 4 may comprise a spring 6 attached to and stretched between the first member 4 and the second member 5.

A preferred embodiment of the invention comprises a means to prevent the first member 4 from moving from its first position when the needle electrode module is not in proximity to a surface. In a preferred embodiment shown in FIGS. 1-2, said means comprises a mechanical linkage 7 that is moved from a first position out of the path of movement of the first member 4 when a portion of the mechanical linkage 7 that extends below the plane of the base member 2 contacts the surface. However, said means may comprise, for example, an electronic sensor that detects the surface and electro-mechanical device that moves out of the path of the first member 4 when the base member 2 is in proximity of the surface. Also in a preferred embodiment, as shown in FIGS. 1-2, the mechanical linkage 7 may be positioned so that when the second member 5 is moved to its second position the mechanical linkage 7 is deformed and said deformation results in the mechanical linkage 7 exerting a force on the second member 5 that tends to move the second member 5 from its second position to its first position.

As shown in FIGS. 1-2, in a preferred embodiment the needle electrode module comprises at least a channel 8 in which at least one guide portion 9 of the first member 4 may move such that the channel 8 restricts the first member 4 to movement along a specific path that allows the first member 4 to move at least from its first position to its second position. Further, in a preferred embodiment shown in FIGS. 1-2, the mechanical linkage 7 may comprise a portion that blocks the movement of the first member 4 when the needle electrode module is in proximity to the surface. In the preferred embodiment, the force exerted by the deformation of the mechanical linkage 7 on the second member 5 is sufficient to move the second member 5 from its second position to its first position if a guide portion 9 of the first member 4 is not located in a portion of a channel 8 that prevents such motion. As shown in FIGS. 1-2, the second member 5 may be connected to the base member 2 via at least one cylindrical member 10 that allows movement of the second member 5 about the axis of each cylindrical member 10.

To use the needle electrode module of FIGS. 1-2, the base member 2 is placed against the surface of a patient's skin. Before the needle electrode module of FIGS. 1-2 is placed against a surface, the mechanical linkage 7 prevents a user from accidentally or intentionally moving the first member 4 from its first position to at least a second position. Because the second member 5 cannot move from its first position to its second position, which results in the extension of the needle member 1 below the plane of the base member 2, when the first member 4 is in its first position, the needle member 1 may not be accidentally or intentionally extended so the risk of an accidental needle-stick injury is reduced.

Once the base member 2 is placed against a surface, the adhesive layer 3 holds the needle electrode module in place and a user may move the first member 4 shown in FIGS. 1-2 from its first position to its second position. Then, the user may move the second member 5 from its first position to its second position, thereby extending the distal end of the needle member 1 below the plane of the base member 2 and through the skin of the patient and into underlying tissue. When the user moves the first member 4 from its first position to its second position, the path of motion of the first member 4 is defined by the interaction of the guide portion 9 of the first member 4 and the channel 8 and the path of movement of the needle member 1 is generally that of an arc about the axis of the cylindrical member 10 that attaches the second member 5 to the base member 2. At this time, the mechanical linkage 7 is deformed and exerts a force on the second member 5 that tends to move the second member 5 from its second position to its first position.

When the second member 5 is in its second position, the force of the spring 6 moves the first member 4 from its second position along a path defined by the channel 8 to a position in which the first member 4 contacts a portion of the mechanical linkage 7 and further motion of first member 4 as the result of the force exerted by the spring 6 is stopped. As shown in FIGS. 1-2, the first member 4 and second member 5 are connected such that when the first member 4 is in this position, the second member 5 may not move from its second position. However, when the needle electrode module is removed from the surface, the mechanical linkage 7 returns to its first position in which it does not restrict the movement of the first member 4 in response to the force of the spring 6. Thus, the force of the spring 6 acts to move the first member 4 so that the guide portion 9 of the first member 4 is in a position such that the first member 4 does not restrict the movement of the second member 5 from its second position to its first position.

When the guide portion 9 of the first member 4 is in a position such that the first member 4 dose not restrict the movement of the second member 5 from its second position to its first position, the force of the mechanical linkage 7 on the second member 5 as a result of the deformation of the mechanical linkage 7 acts to return the second member 5 to its first position from its second position. Finally, as shown in FIGS. 1-2, in a preferred embodiment the channel 8 defines a last position into which the first member 4 may move when the second member 5 returns to its first position and, in said last position of the first member 4, the second member 5 is prevented from moving from the first position of the second member 5.

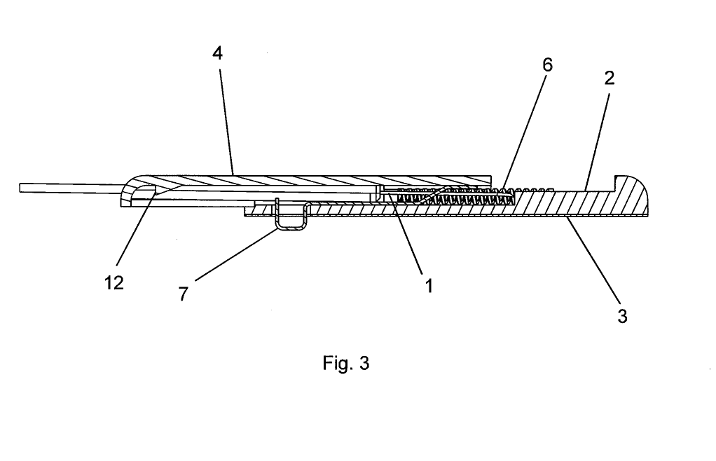

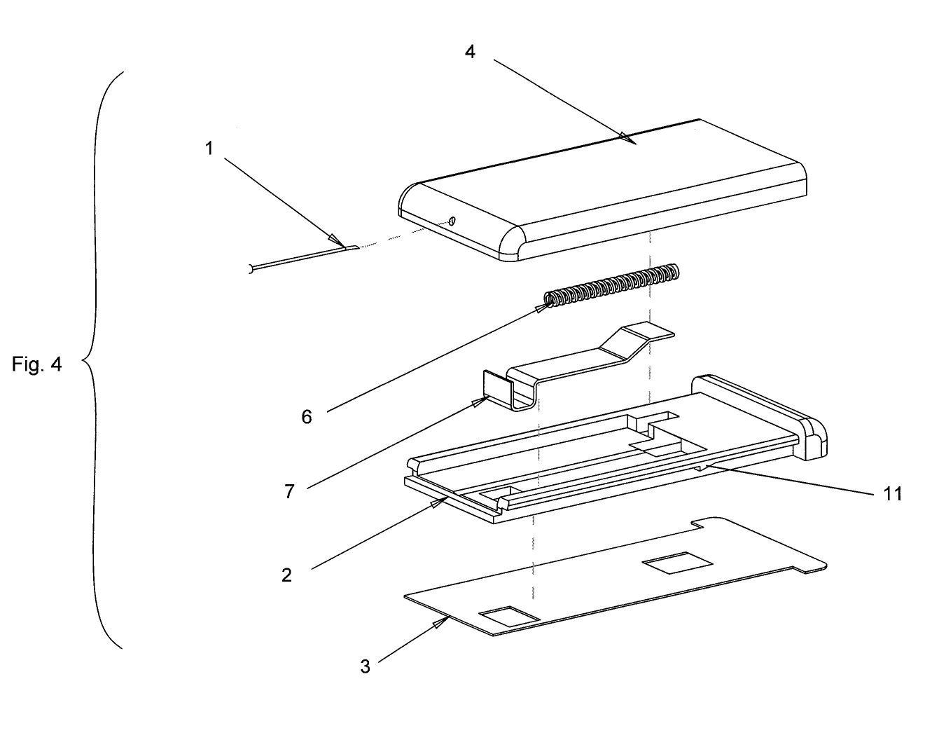

A second embodiment of the invention is shown in FIGS. 3-4. In this embodiment, the distal end of the needle member 1 is extended below the plane of the base member 2 by moving the first member 4 from its first position to its second position. When the needle electrode module is not in proximity of a surface, the first member 4 cannot be moved from its first position due to a first catch mechanism 11 of the base member 2 shown in FIG. 4 that prevents movement of first member 4 when the base member 2 is not in proximity of a surface.

The first catch mechanism 11 of the second embodiment shown in FIG. 4 prevents movement of the first member 4 when the base member 2 is not in proximity of a surface and the mechanical linkage 7 is in a first position. When the mechanical linkage 7 of FIGS. 3-4 is in its first position, a first end of the mechanical linkage 7 extends below the plane of the base member 2 and a second end of the mechanical linkage 7 applies a force to the first member 4 that maintains contact between a first catch portion of the first member 4 and the first catch mechanism 11. When the base member 2 of FIGS. 3-4 is in proximity of a surface, the first end of the mechanical linkage 7 is moved to a second position of the mechanical linkage 7. In the second position of the mechanical linkage 7, the first end of the mechanical linkage 7 extends to roughly the plane of the base member 2 and the force applied by the second end of the mechanical linkage 7 to the first member 4 is lessened such that the contact between the first catch portion of the first member 4 and first catch mechanism 11 is lessened such that the first member 4 may move relative to the base member 2.

In the second embodiment, once the mechanical linkage 7 is in its second position and the first member 4 is moved to its second position, a second catch portion 12 of the first member 4, shown in FIG. 3, contacts a portion of the mechanical linkage 7. As long as the second catch portion 12 is in contact with the mechanical linkage 7, the first member 4 is prevented from returning to its first position. When the base member 2 shown in FIGS. 3-4 is removed from the proximity of a surface, the mechanical linkage 7 returns to its first position and the first member 4 can move back to its first position.

The second embodiment further comprises a means for applying a force to the first member 4 that tends to move the first member 4 from its second position to its first position.

In a preferred embodiment the means of applying force to the first member 4 comprises a spring 6 shown in FIGS. 3-4 located between the first member 4 and base member 2 such that the spring 6 applies said force to the first member 4. As a result of the force applied to the first member 4, when the first member 4 is in its second position and the mechanical linkage of FIGS. 3-4 returns to its first position when the base member 2 is removed from the proximity of the surface, the first member 4 is automatically moved to its first position.

In a preferred embodiment, movement of the first member 4 in FIGS. 3-4 from its first position to its second position causes the needle member 1 to move along a guide portion of the needle electrode module that results in the extension of the needle member 1 below the plane of the base member 2 at a desired angle.

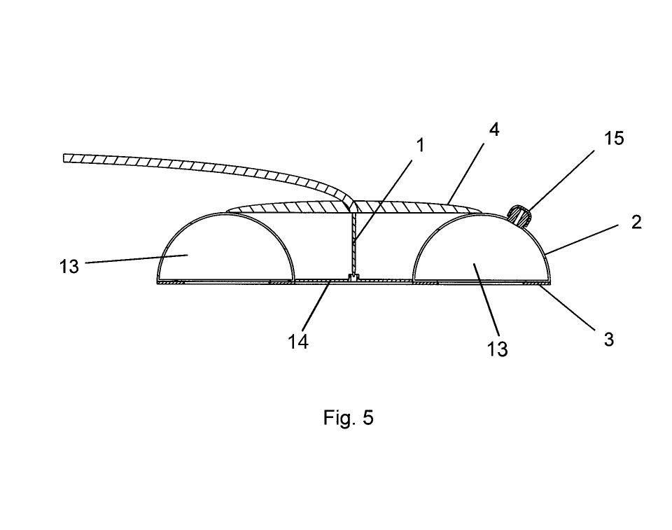

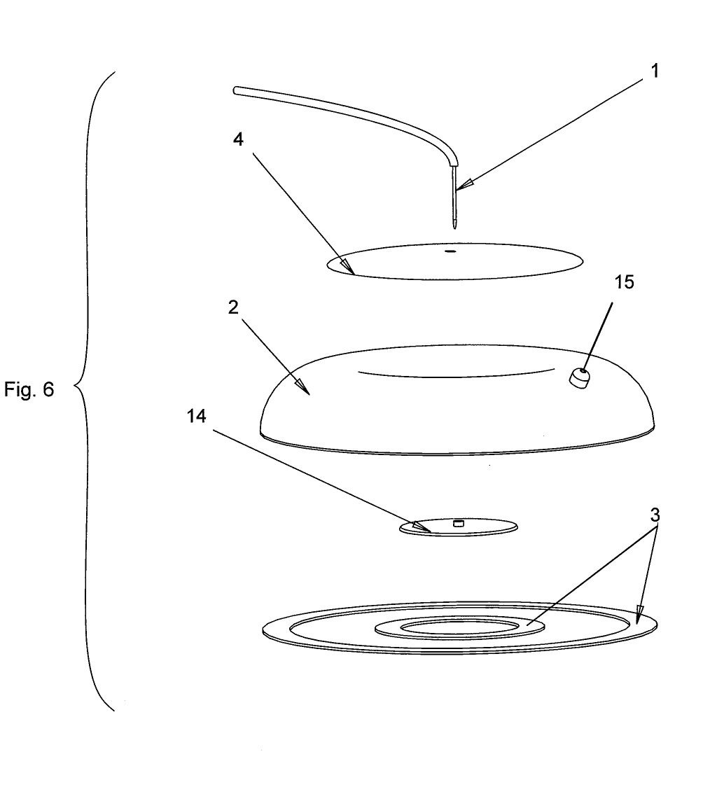

FIGS. 5-6 show another embodiment of this invention. The needle electrode module comprises a base member 2 comprising a flexible material that resists deformation and that, after being deformed, will return generally to its pre-deformation shape. For example, common plastic or rubber compounds have the requisite properties. In this embodiment, base member 2 further comprises a shape that forms a cavity 13 when the base member 2 is in proximity of a surface. The base member 2 also comprises at least one valve 15 that allows the movement of air in only one direction from inside the cavity 13 formed by the base member 2 to the area outside the cavity 13. In a preferred embodiment the base member 2 is generally toroidal in shape. This embodiment of the invention further comprises an adhesive layer 3 affixed to the portion of the base member 2 that is to be located in proximity to the surface.

In addition, this embodiment comprises a first member 4 attached to the base member 2 and needle member 1. In a preferred embodiment, the first member 4 comprises a material more rigid than the base member 2 and is attached to the base member 2 such that a force applied to the first member 4 may be distributed across the base member 2. A guide member 14 directs the needle member 1 to extend below the plane of the base member 2 at a desired angle.

To use the embodiment of FIGS. 5-6, the base member 2 is located in proximity to a surface and its position in proximity to the surface is secured by the adhesive layer 3. When so located and secured, the base member 2 forms a cavity 13 substantially sealed from the area outside the cavity 13 and that defines a first volume of air that may communicate with the area outside the cavity 13 via the valve 15. A force applied by a user to the first member 4 causes the base member 2 to deform such that the cavity 13 defines a second volume, said second volume being less than the first volume. When the volume of the cavity 13 is reduced from the first volume to the second volume, air moves from the cavity 13 through the valve 15 such that the air in the cavity 13 is generally in equilibrium with the air outside the cavity 13. Deformation of the base member 2 results in the movement of the needle member 1 from its first position to its second position in which the distal end of the needle member 1 extends below the plane of the base member 2.

When the user-applied force is removed from the first member 4, the material of the base member 2 will act to return the base member 2 to generally its pre-deformation shape, however this action will create a vacuum or suction force in the cavity 13 that will prevent the base member 2 from returning generally to its pre-deformation shape. Thus, the needle member 1 will remain in its second position until the base member 2 is removed from the proximity of the surface, at which time the cavity 13 formed by the base member 2 will be open to the atmosphere and the vacuum or suction force will no longer exist. Without the vacuum or suction force acting on the base member 2, the base member 2 will not be prevented from returning generally to its pre-deformation shape so the base member 2 will return generally to its pre-deformation shape and the needle member 1 will return to its first position.

What is claimed is:

1. A needle electrode module comprising:
a base member having a substantially planar surface adapted to be brought into proximity to a surface;
a needle member having at least a first and second position; wherein in the first position of the needle member the distal end of the needle member does not extend below the plane of the base member; and in the second position of the needle member the distal end of the needle member extends retractably below the plane of the base member;
a means for electrically connecting the needle member to an electrical source;
a means for securing the needle member in its first position when the base member is not in the proximity of the surface and allowing the needle member to move into and stay in its second position when the base member is in the proximity of the surface; and
a means for securing the base member in the proximity of the surface.

2. The needle electrode module of claim 1 wherein the means for securing the base member in the proximity of the surface is an adhesive.

3. The needle electrode module of claim 1 wherein the means for securing the base member in the proximity of the surface is the application of suction to the surface.

4. The needle electrode module of claim 1 wherein the means for securing the needle member in its first position when the base member is not in the proximity of the surface and allowing the needle member to move into and stay in its second position when the base member is in the proximity of the surface comprises:
a first member having at least a first and second position;
a means for preventing the first member from moving from its first position when the base member is not in proximity of the surface and allowing the first member to move from its first position when the base member is in proximity of the surface;
a second member having at least a first and second position; wherein the second member is attached to the needle electrode module such that the second member cannot cause the distal end of the needle member to extend below the plane of the base member when the second member is its first position and the second member causes the needle member to be extended below the plane of the base member when the second member is its second position; the second member cannot be moved from its first position when the first member is in its first position; and the second member may be moved into its second position when the first member is in its second position; and
a means for applying a first force to the second member that tends to move the second member from its second position to its first position.

5. The needle electrode module of claim 4 wherein the first member has a last position into which it may be moved after its second position and while the needle member is extended below the plane of the base member; while the first member is its last position the needle member may be retracted such that it does not extend below the plane of the base member; and while the first member is in its last position, once the needle member is retracted, the needle member may not be extended below the plane of the base member.

6. The needle electrode module of claim 5 wherein the first member may not be accidentally moved from its last position.

7. The needle electrode of claim 4 wherein the first member comprises a plurality of guides; the needle electrode comprises a plurality of channels such that each guide fits into and may move within at least one channel; and each channel, through interaction with a guide, restricts the first member to a path of movement that allows the first member to move from its first position to at least its second position.

8. The needle electrode of claim 7 further comprising a means for applying a second force to the first member, said second force tending to move the first member from its second position to at least its first position.

9. The needle electrode of claim 8 wherein the second force is applied to the first member by a spring.

10. The needle electrode of claim 7 wherein at least the portion of a channel corresponding to the second position of the first member is generally perpendicular to the second force such that when a guide is in this portion of the channel the first member may not be moved by the second force.

11. The needle electrode module of claim 1 wherein the means for securing the needle member in its first position when the base member is not in the proximity of the surface and allowing the needle member to move into and stay in its second position when the base member is in the proximity of the surface comprises:
a first member having at least a first and second position;
a means for preventing the first member from moving from its first position when the base member is not in proximity of the surface and allowing the first member to move from its first position when the base member is in proximity of the surface;
a means for preventing the first member from moving from its second position to its first position when the base member is in proximity of the surface;
a guide that directs the distal end of the needle member to extend below the plane of the base member at desired angle; and
a means for applying a first force to the first member that tends to move the first member from its second position to its first position.

12. The needle electrode module of claim 11 wherein the means for preventing the first member from moving from its first position when the base member is not in proximity of the surface and allowing the first member to move from its first position when the base member is in proximity of the surface comprises:
a mechanical linkage having at least a first position and a second position and comprising a first end and a second end wherein the mechanical linkage is in its first position when the base member is not in proximity of the surface, and when in said first position, the first end of the mechanical linkage extends below when the base member and the second end of the mechanical linkage applies a force to the first member; and wherein the mechanical linkage is in its second position when base member is in proximity of the surface, and when in said second position, the first end of the mechanical linkage extends to roughly the plane of the base member and the second end of the mechanical linkage applies less force to the first member; and
a catch mechanism that prevents the first member from moving relative to the base member when the mechanical linkage is in its first position; said catch mechanism releasing the first member to move relative to the second member when the mechanical linkage is in its second position.

13. The needle electrode modules of claim 11 or 12 wherein the means for preventing the first member from moving from its second position to its first position when the base member is in proximity of the surface comprises:
catch mechanism on the first member;
a mechanical linkage having at least a first position and a second position and comprising a first end wherein the mechanical linkage is in its first position when the base member is not in proximity of the surface, and when in said first position, the first end of the mechanical linkage extends below when the base member and the mechanical linkage does not contact the catch mechanism on the first member;
wherein the mechanical linkage is in its second position when base member is in proximity of the surface, and when the mechanical linkage is in its second position and the first member is in its second position, the first end of the mechanical linkage extends to roughly the plane of the base member and a portion of the mechanical linkage contacts the catch mechanism on the first member and prevents the first member from returning to the first position of the first member; and wherein when the mechanical linkage returns to its first position it no longer contacts the catch mechanism on the first member and the first member is not prevented from returning to its first position.

14. The needle electrode module of claim 3 wherein:
the base member comprises a flexible material that resists deformation such that when the base member is deformed it will return generally to its pre-deformed shape unless a force acts upon the base member to prevent said return;
the base member forms an air-filled cavity when in proximity of the surface and wherein the volume of air defined by said cavity is in communication with air outside the cavity through at least one valve that allows air to move in only one direction from the cavity to the area outside the cavity;
the resistance of the base member to deformation results in a suction force being applied to the surface when the volume is reduced through deformation of base member and wherein the suction force is strong enough to prevent the base member from returning generally to its pre-deformed shape and to hold the needle member in place;
the needle member is attached to the base member such that the needle is in its first position when the base member is not deformed and the needle member is in its second position when the base member is deformed, and wherein the resistance of the base member to deformation comprises the means for securing the needle member in its first position and allowing the needle member to move into and stay in its second position; and
removal of the base member from the surface when the base member is deformed eliminates the suction force and allows the base member to return generally to its pre-deformed shape in which the needle in is its first position.
